(19) United States
(12) Patent Application Publication
(10) Pub. No.: US 2010/0240753 A1
(43) Pub. Date: Sep. 23, 2010

(54) EFFECTIVE PHARMACEUTICAL CARRIER FOR POORLY BIOAVAILABLE DRUGS

(76) Inventors: Bee Hong Ng, Pulau Pinang (MY); Wai Peng Choy, Perak (MY); Jia Woei Wong, Pulau Pinang (MY); June Lay Choo Khoo, Perak (MY); David Sue San Ho, Perak (MY); Kah Hay Yuen, Pulau Pinang (MY)

Correspondence Address: MEYER UNKOVIC & SCOTT LLP, 535 Smithfield Street, Suite 1300, Pittsburgh, PA, 15222-2315, US

(21) Appl. No.: 12/308,827

(22) PCT Filed: Jun. 15, 2007
(86) PCT No.: PCT/MY2007/000040

§ 371 Date: Dec. 24, 2008

(30) Foreign Application Priority Data
Jun. 28, 2006 (MY) PI 20063061

(51) Int. Cl. A61K 31/201; A61K 31/20; A61K 31/19

(52) U.S. Cl. 514/560; 514/558; 514/557

(57) ABSTRACT

The present invention is directed to an improved effectiveness pharmaceutical carrier comprising anyone or a combination of edible or pharmaceutical acceptable fatty acids and anyone or a combination of non-ionic surfactants, which is capable of improving the bio-absorption of drugs with intermediate log P ranging from 2 to 4 (having poor solubility in both water and triglycerides) as well as those with high log P of more than 4.

### FIELD OF INVENTION

The present invention relates to an improved effectiveness pharmaceutical carrier prepared for enhancing the oral bioavailability of drugs with intermediate log P of 2 to 4 (having limited solubility in both water and triglycerides) as well as those with high log P of more than 4.

### BACKGROUND OF THE INVENTION

Emulsions have been known to improve absorption of oil-soluble drugs such as griseofulvin (Carrigan and Bates, 1973; Bates and Carrigan, 1975; Bates and Sequeira, 1975), phenytoin (Chakrabati and Belpaire, 1978) and danazol (Charman et al., 1993). However, conventional emulsions are not well accepted due to their bulkiness, shorter shelf-life and poor palatability. Therefore, self-emulsifying drug delivery systems (SEDDSs) have gained increasing interests in recent years due to many advantages they have over conventional emulsions. According to the definition by Pouton (1985 and 1997), a self-emulsifying system is an isotropic mixture of oil and surfactant(s), sometimes including co-solvent(s), which emulsifies spontaneously or under conditions of gentle agitation upon contact with an aqueous medium. Moreover, recent advances in pharmaceutical engineering have led to more affordable encapsulation facilities. Thus, SEDDS can now be prepared in soft-gelatin capsules, which provide better ease and convenience of administration. Upon dissolution of the capsule shell following oral ingestion, the contents faun an emulsion spontaneously or under conditions of gentle agitation after coming into contact with the gastrointestinal fluids. This would in turn lead to improved absorption or bioavailability of the contained drugs.

Several SEDDSs have been patented for the formulation of oil-soluble drugs (U.S. Pat. No. 5,858,401, U.S. Pat. No. 5,965,160; U.S. Pat. No. 6,057,289; U.S. Pat. No. 6,316,497; U.S. Pat. No. 6,436,430; U.S. Pat. No. 6,555,558; U.S. Pat. No. 6,638,522; U.S. Pat. No. 6,960,563; Patent no. WO9929300; Patent no. WO9929316; Patent no. WO9956727) and have been claimed to improve the oral absorption of these drugs (U.S. Pat. No. 5,993,858; U.S. Pat. No. 6,008,192; U.S. Pat. No. 6,056,971; U.S. Pat. No. 6,121,313; U.S. Pat. Nos. 6,231,887; 6,531,139; U.S. Pat. No. 6,596,306; U.S. Pat. No. 6,960,563; U.S. Pat. No. 6,962,931; Patent no. WO9906024). These patented SEDDSs usually involves the use of either mono, di or triglycerides of long or medium chain fatty acids such as monoolein, diolein, triolein and the vegetable oils as well as their ester foul's to dissolve the oil soluble drugs, in combination with suitable surfactant systems. The use of oleic acid as part of the pharmaceutical miler was disclosed in the U.S. Pat. No. 6,057,289, Patent no. WO0066140, Patent no. WO9943299, New Zealand Patent no. NZ528741 and Patent application no. WO2004052405.

On the other hand, there are a few patented systems which required the use of hydrophilic co-solvents, primarily alcohol such as ethanol and propylene glycol (U.S. Pat. No. 6,008,192; U.S. Pat. No. 6,531,139; U.S. Pat. No. 6,960,563; Patent no. WO9929300, Patent no. WO9943299). The use of ethanol is unfavourable mainly due to religious constraints, while propylene glycol has been prohibited by many regulatory bodies because of safety and toxicity on long term ingestion. Meanwhile, U.S. Pat. No. 6,316,497 by Liu and Wang disclosed the use of as high as 15% w/w water in their formulation of SEDDS which is unsuitable for softgel encapsulation. Besides that, some of these patents entailed high concentration of surfactants (U.S. Pat. No. 5,858,401; U.S. Pat. No. 6,008,192; U.S. Pat. No. 6,056,971; U.S. Pat. No. 6,057,289; U.S. Pat. No. 6,638,522) to result in solubilized systems with nano-size droplets upon contact with aqueous solutions. However, these SEDDSs were only designed for poorly bioavailable oil-soluble drugs, but not applicable to those having poor or limited solubility in the glycerides especially triglyceride oil carriers due to low solvent capacity for these groups of drugs.

Among the patents relating to the use of fatty acids as part of the pharmaceutical carrier, Patent no. WO9943299 and Patent application no. WO2004052405 were filed for the ability of their systems in providing protection to poorly absorbable hydrophilic bio-molecules (e.g. peptides), which are susceptible to degradation by the harsh gastrointestinal environment. Meanwhile, only New Zealand Patent no. NZ528741 was related to inventing self-emulsifying pharmaceutical carrier for poorly water-soluble drugs, but without using any conventional surfactants. However, the absence of surfactants was compensated with the use of co-solvents or adjuvants, namely glycols, glycol ethers and organic amines to achieve emulsification. The utilization of these compounds for oral consumption has always been unwarranted. Moreover, the fatty acids recommended were only limited to those having 6 to 18 carbon atoms.

The prior arts of U.S. Pat. No. 6,057,289 and Patent no. WO0066140 disclosed a pharmaceutical composition comprising a pharmaceutically effective amount of cyclosporine in association with a pharmaceutical carrier, said carrier comprising (a) a cyclosporine solubilizing agent consisting essentially of an effective amount of a fatty acid of 6 to 22 carbon atoms, and (b) a non-ionic surfactant having an HLB value greater than 10, said non-ionic surfactant being present with the cyclosporine solubilizing agent and cyclosporine in an amount sufficient to foul an emulsion when brought into contact with an aqueous medium in a mammal. Its field of invention only relates to a pharmaceutical carrier system developed specifically for cyclosporine in which the solubility of cyclosporine was enhanced in the said pharmaceutical carrier. The fatty acid, namely oleic acid, was chosen for its similar lipophilicity to cyclosporine. Besides that, like most SEDDS, the said pharmaceutical carrier was found to be effective only if the surfactant concentration utilized was more than 50% by weight, with the preferred ratio of oleic acid to non-ionic surfactant ranging from 1:1 to 1:4 w/w.

The present invention led to the discovery of using a pharmaceutical carrier comprising only anyone or a combination of edible or pharmaceutically acceptable fatty acids and anyone or a combination of non-ionic surfactants to deliver a wide range of drugs, encompassing those having intermediate log P (being poorly soluble in both water and triglycerides) as well as high log P. Unlike the invention revealed in U.S. Pat. No. 6,057,289 and Patent no. WO0066140, the preferred ratio of fatty acid to non-ionic surfactant used in the present improved effectiveness pharmaceutical carrier was found to be 9:1 w/w, with minimal use of non-ionic surfactant, i.e. 10% by weight. Thus, besides preventing long term ingestion of high amount of surfactants, the present improved effectiveness pharmaceutical carrier also does not use any co-solvents or adjuvants (e.g. glycols, glycol ethers and organic amines). More importantly, the present improved effectiveness pharmaceutical carrier is also able to enhance the bio-absorption of a wide range of drugs with intermediate log P (having poor solubility in both water and triglycerides) as well as those with high log, P, following oral administration. It is therefore apparent that discoveries of the present invention were not obvious in any prior arts related to self-emulsifying pharmaceutical carriers, whereby majority of them were only limited for the formulation of oil-soluble' drugs (with high log P of more than 4). Moreover, findings from the present improved effectiveness pharmaceutical carrier were not obvious in the prior arts of U.S. Pat. No. 6,057,289 and Patent no. WO0066140 which disclosed a similar pharmaceutical carrier, meant only to dissolve cyclosporine without any proof of improved bioavailability.

### SUMMARY OF THE INVENTION

The present invention advantageously provides an improved effectiveness pharmaceutical carrier comprising anyone or a combination of edible or pharmaceutically acceptable fatty acids and anyone or a combination of non-ionic surfactants to deliver orally ingested drugs.

It is also an objective of the present invention that the said improved effectiveness pharmaceutical carrier comprising anyone or a combination of edible or pharmaceutically acceptable fatty acids and anyone or a combination of non-ionic surfactants, is capable of enhancing bio-absorption of a wide range of orally ingested drugs, with intermediate log P of 2 to 4 (being poorly soluble in both water and triglycerides) as well as those with high log P of more than 4.

### BRIEF DESCRIPTION OF THE DRAWING

FIG. 1 is a graph showing mean plasma ubiquinone concentration versus time profiles of the reference product and the preparation in disclosed pharmaceutical carrier

FIG. 2 is a graph showing the extent of absorption of ubiquinone (cumulative AUC) of the reference product and the drug prepared in disclosed pharmaceutical carrier at day 1, 3 and 7 (n=6)

### DETAILED DESCRIPTION OF THE INVENTION

The term “carrier” is a term of art. As used, herein, the term “carrier” refers to the composition that transports the drug across the biological membrane or within a biological fluid. The present invention provides an improved effectiveness pharmaceutical carrier for SEDDS based formulation comprising,
anyone or a combination of fatty acids, and
anyone or a combination of non-ionic surfactants.

The disclosed carrier is preferably utilized to deliver pharmaceutical active agents having intermediate log P of 2 to 4 (with limited solubility in both water and triglycerides) as well as those having high log P of more than 4.

The first component, as indicated hereinabove, is a fatty acid or a mixture of different types of edible and pharmaceutically acceptable fatty acids. The fatty acids mentioned hereinabove are preferred to be saturated or unsaturated fatty acids with carbon chain range from C₁₂ to C₂₂. Representative examples of these fatty acids are oleic acid, eleostearic acid, lauric acid, myristic acid, palmitic acid, stearic acid, elaidic acid, linoleic acid, linolenic acid, and docosahexaenoic acid. Among all the fatty acids, oleic acid is most preferred due to its excellent solvent capacity and its additional capability in reducing cholesterol level in blood.

The abovementioned non-ionic surfactant or the combination of more than one type of surfactants is preferably to have hydrophile-lipophile balance (HLB) value ranging between 11 and 17 to achieve the optimum effect of said pharmaceutical carrier. In the preferred embodiment of the present invention, such non-ionic surfactants are selected from a group of representative non-ionic surfactants which include, polyoxyethylene (20) sorbitan monooleate, polyoxyethylene (20) sorbitan monostearate, glyceryl polyethylene glycol oxystearate (Cremophor® CO and RH grades), glycerol polyethylene glycol ricinoleate (Cremophor® EL), sucrose stearate, sucrose oleate, sucrose palmitate, sucrose myristate, sucrose laurate, decaglycerol lauric acid esters, decaglycerol myristic acid esters, decaglycerol stearic acid esters. For example, using glycerol polyethylene glycol ricinoleate (Cremophor® EL) in the carrier has enabled the said improved effectiveness pharmaceutical carrier to self-emulsify easily in an aqueous environment with gentle agitation.

In the preferred embodiment of the present invention, the abovementioned fatty acids and non-ionic surfactant are mixed in a ratio ranging from 9.5:0.5 w/w to 1:1 w/w to form the said improved effectiveness pharmaceutical carrier and the most preferred ratio is 9:1 w/w. This improved effectiveness pharmaceutical carrier is readily to be filled into soft gelatin capsule (or capsules made of gel-forming materials such as starches, polymers, cellulose or its derivatives) with the preferred medication, which disintegrates and releases the contents, and subsequently fowls an emulsion.

The drug mentioned hereinabove may be suitably used within a range of amount which can express its therapeutic effect, according to the disease to be treated, age, weight, nature and condition of the patient, to be treated. The disclosed improved effectiveness pharmaceutical carrier in the present invention is an excellent carrier for medications with limited solubility in both water and triglycerides as mostly represented by medications with intermediate log P (partition coefficient) values ranging from 2 to 4. Medications of low solubility in both water and triglycerides include, but not limited to, griseofluvin (2.18), pravastatin (2.42), carbamazepine (2.45), phenytoin (2.47), piroxicam (3.06); ketoprofen (3.12), naproxen (3.18), testosterone (3.22), progesterone (3.87), and ibuprofen (3.97). In another preferred embodiment, the disclosed improved effectiveness pharmaceutical carrier is also suitable to perform as carrier for medications with log P (partition coefficient) more than 4. Examples for said medication include, but not limited to, lovastatin (4.26), indomethacin (4.27), ketoconazole (4.35), diclofenac (4.51), simvastatin (4.68), gemfibrozil (4.77), testosterone undecanoate (8.77) and ubiquonone (more than 10).

The improved effectiveness pharmaceutical carrier was prepared by mixing anyone or a combination of fatty acids and anyone or a combination of non-ionic surfactants mentioned hereinabove, in a ratio within the range of 9.5:0.5 w/w to 1:1 w/w. For example, to prepare a 100 g of formulation for ubiquinone, 6 g of ubiquinone is required to mix with 94 g (84.6 g of fatty acids and 9.4 g of non-ionic surfactant) of carrier until the drug fully dissolves.

The following example using ubiquinone as one of the said drugs is intended to further illustrate the capability of the present invention in enhancing bio-absorption of poorly bioavailable drugs having log P values more than 2, in healthy human volunteers; without any intent for the invention to be limited to the specific embodiments described herein.

### Example

A comparative in vivo bioavailability study was conducted to investigate the bioavailability of ubiquinone prepared in the disclosed pharmaceutical carrier compared to that of the reference product. The reference product was conventional formulation comprising ubiquinone in soybean oil. Both products were prepared in the form of gelatine capsules. Six healthy adult male volunteers participated in the two way crossover study after providing informed consent. The volunteers were randomly divided into 2 groups of 3 each, and administered the preparations according to the schedule shown in Table 1.

TABLE 1

| | Period | |
|---|---|---|
| Group | I | II |
| 1 | Reference Product | Drug Prepared in Disclosed |
| | | Pharmaceutical Carrier |
| 2 | Drug Prepared in Disclosed | Reference Product |
| | Pharmaceutical Carrier | |

On the first trial period, each volunteer in group 1 was given 6 capsules of the reference product, while those in group 2, 6 capsules of same drug prepared in the disclosed pharmaceutical carrier containing an equivalent dose of ubiquinone were administered to each volunteer. After a wash-out period of 3 weeks, each volunteer then received the alternate product.

All products were administered with 240 ml of water in the morning after an overnight fast of 12 hours. Food and beverages were withheld for at least 4 hours after dosing and plain water given ad libitum one hour after dosing. Lunch and dinner were served at 4 and 10 hours after dosing. Blood samples of 7-ml volume were collected in vacutainers (containing sodium heparin as anticoagulant) at 0 (before dosing), 2, 4, 6, 8, 10, 12, 14, 18, 24 hours via an in-dwelling cannula placed in the forearm. Blood samples at 30, 36, 48, 60, 72, 96 and 144 hours were collected via venipuncture. The blood samples were centrifuged for 15 minutes at 2000 g and the plasma transferred to separate glass containers to be kept frozen until, analysis.

Plasma levels of ubiquinone were analysed using a validated reversed-phase high performance liquid chromatographic method.

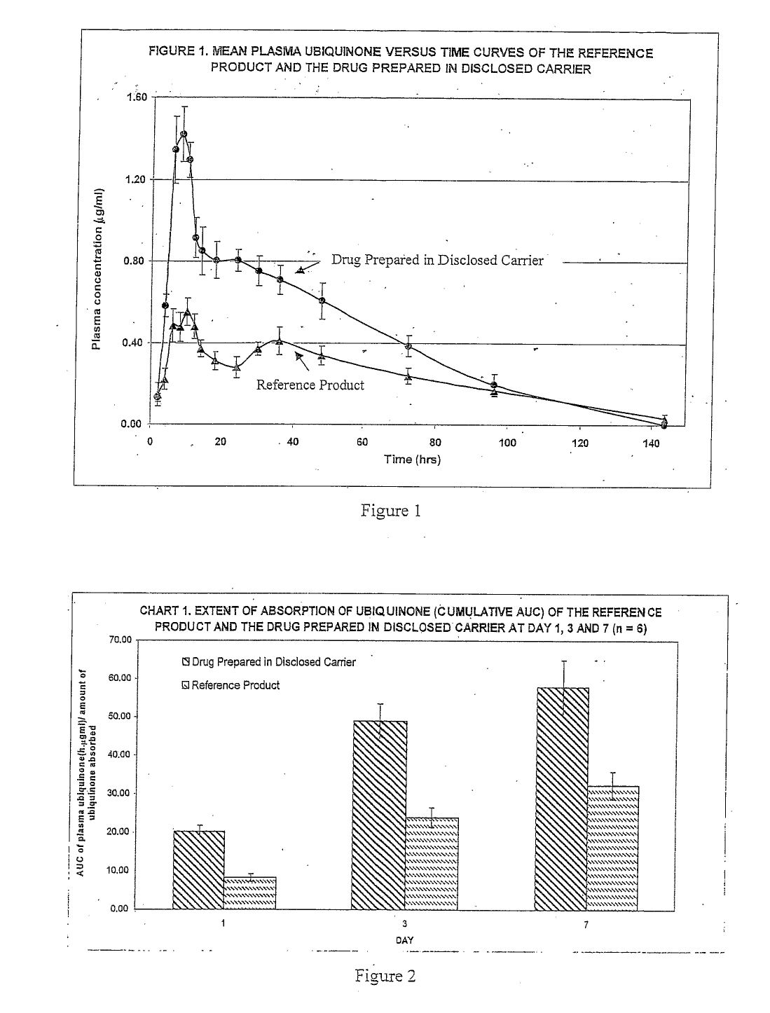

The mean plasma ubiquinone concentration versus time profiles of the reference product and the preparation in disclosed pharmaceutical carrier are shown in FIG. 1. It is apparent that the plasma profile of the drug prepared in the disclosed pharmaceutical carrier was higher than that of the reference product. Also, the initial rapid increase in plasma ubiquinone concentrations of the drug prepared in the disclosed pharmaceutical carrier indicates ubiquinone is more efficiently absorbed from the disclosed pharmaceutical carrier compared to the reference product.

The extent of absorption of ubiquinone as represented by cumulative mean area under the plasma ubiquinone concentration-time curve (AUC) at day 1, 3 and 7 of the reference products and the drug prepared in disclosed carrier is shown in Chart 1. The chart clearly depicts that the ultimate amount of ubiquinone absorbed from the disclosed pharmaceutical carrier was almost double of that of the reference product at day 7 although the same quantity of ubiquinone was administered. This clearly explains the enhanced bio-absorption of ubiquinone as a result of the more efficient absorption from the disclosed pharmaceutical carrier compared to the reference product.

Besides that, there was a statistically significant difference between the drug prepared in the disclosed pharmaceutical carrier and the reference product with respect to both logarithmic transformed values of AUC₀₋₁₄₄ₕ (p<0.05) as well as Cₘₐₓ (p<0.05). From the statistical analysis of AUC₀₋₁₄₄ₕ values, it was also estimated that the extent of absorption of the drug prepared in the disclosed pharmaceutical carrier was approximately 2 times higher than that of the reference product.

It is to be understood that the present invention may be embodied in other specific forms and is not limited to the sole embodiment described above. However modification and equivalents of the disclosed concepts such as those which readily occur to one skilled in the art are intended to be included within the scope of the claims which are appended thereto.

1. A pharmaceutical carrier for SEDDS based formulation comprising:
(a) an edible and pharmaceutically acceptable fatty acid; and
(b) a non-ionic surfactant.

2. A pharmaceutical carrier of claim 1, wherein the fatty acid has a saturated or unsaturated C₁₂-C₂₂ carbon chain.

3. A pharmaceutical carrier of claim 1, wherein the fatty acid includes, anyone or a combination of oleic acid, eleostearic acid, lauric acid, myristic acid, palmitic acid, stearic acid, elaidic acid, linoleic acid, linolenic acid, and docosahexaenoic acid.

4. A pharmaceutical carrier of claim 1, wherein the non-ionic surfactant includes, anyone or a combination of polyoxyethylene (20) sorbitan monooleate, polyoxyethylene (20) sorbitan monostearate, glyceryl polyethylene glycol oxystearate (Cremophor® CO and RH-grades), glycerol polyethylene glycol ricinoleate (Cremophor® EL), sucrose stearate, sucrose oleate, sucrose palmitate, sucrose myristate, sucrose laurate, decaglycerol lauric acid esters, decaglycerol myristic acid esters, decaglycerol stearic acid esters.

5. A pharmaceutical carrier of claim 1, wherein the surfactant has a hydrophile-lipophile balance (HLB) value ranging from 11 to 17.

6. A pharmaceutical carrier of claim 1, wherein the pharmaceutically acceptable fatty acid and non-ionic surfactant are mixed in a ratio ranging from 9.5:0.5 w/w to 1:1 w/w.

7. A pharmaceutical carrier of claims 1 to 6 is used to dissolve and improve the bio-absorption of drugs with limited solubility in both water and triglycerides with intermediate log P (partition coefficient) ranging from 2 to 4.

8. A pharmaceutical carrier of claims 1 to 6 is used to dissolve and improve the bio-absorption of drugs with high log P (partition coefficient) of more than 4.
